(19) United States
(12) Patent Application Publication
(10) Pub. No.: US 2023/0301993 A1
(43) Pub. Date: Sep. 28, 2023

(54) Low-Dose Ophthalmic Compositions and Methods

(71) Applicant: Vyluma Inc., Bridgewater, NJ (US)

(72) Inventors: Jack Martin Lipman, West Milford, NJ (US); Kumaresh Soppimath, Skillman, NJ (US); Tushar Hingorani, Bridgewater, NJ (US)

(73) Assignee: Vyluma Inc., Bridgewater (US)

(21) Appl. No.: 18/019,655

(22) PCT Filed: Jul. 23, 2021
(86) PCT No.: PCT/US2021/043061

§ 371 Date: Feb. 3, 2023

Related U.S. Application Data
(60) Provisional application No. 63/068,516, filed on Aug. 21, 2020.

(51) Int. Cl. A61K 31/498; A61K 31/27; A61K 31/55; A61K 31/221; A61K 9/00; A61P 27/02

(52) U.S. Cl. CPC A61K 31/498; A61K 31/27; A61K 31/55; A61K 31/221; A61K 9/0048; A61P 27/02

(57) ABSTRACT

Ophthalmic formulations for treatment of night vision disturbance syndrome (NVD) are presented and preferably comprise brimonidine, rivastigmine, and/or galantamine at very low concentrations. Such formulations unexpectedly provided acute and transient therapeutic effect to alleviate one or more symptoms associated with NVD.

This application claims priority to our co-pending U.S. Provisional Patent Application with the Ser. No. 63/068,516, which was filed Aug. 21, 2020, and which is incorporated by reference herein.

### FIELD OF THE INVENTION

The field of the invention is topical ophthalmic compositions comprising brimonidine, rivastigmine, and/or galantamine, and methods therefore, especially as it relates to treatment of night vision disturbance syndrome.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Night vision disturbances (NVD), especially after refractive surgery are unfortunately relatively common and adversely affect many ordinary tasks under low-light conditions. Most often, individuals suffering from NVD experience glare, starbursts, double vision, and halo around brighter objects under low-light conditions or darkness. Given the limited time during which an individual experiences NVD, therapeutic interventions should be restricted to those that are acute and transient to provide prompt effect upon administration of a drug, but also of sufficiently short duration so as to not interfere with vision under non-scotopic conditions. A study performed using aceclidine (1-azabicyclo[2.2.2]octan-3-yl acetate), typically used to treat open-angle glaucoma, provided reduction in some symptoms of NVD (see J Cataract Refract Surg. 2005 September; 31(9):1764-72). However, the effect of aceclidine was relatively extended and as such not suitable for acute and transient treatment of NVD. Furthermore, aceclidine is known to have significant side effects, including increased salivation and bradycardia.

In another example, WO 00/64425 describes the use of selected miotic agents, such as certain cholinomimetic active agents and cholinesterase inhibitors, in combination with one or more hypertonic agents, such as sulfacetamide and derivatives thereof to treat visual disorders characterized by reduced contrast sensitivity. Unfortunately, the duration of the drug effect was once more relatively long (up to 14 hours) and thus rendered the formulation unsuitable for acute and transient use. In related examples, use of one or more parasympathomimetic drugs or cholinesterase inhibitors in combination with one or more alpha agonists or antagonists have been described to temporarily treat presbyopia as can be taken from U.S. Pat. No. 8,455,494 and WO 2010/135731. However, such formulations have not been proven effective or even considered to treat NVD.

Brimonidine, an alpha-2 agonist, is disclosed in U.S. Pat. Nos. 3,890,319, and 5,856,329. WO 2004/043933 teaches use of brimonidine for treatment of glaucoma. Similarly, US 2004/0102445 teaches use of a combination of brimonidine and timolol for treatment of glaucoma. In further known ophthalmic uses, U.S. Pat. Nos. 6,194,415 and 6,248,741 describe the use of brimonidine for providing neuroprotection to the eye. Rivastigmine is a known selective inhibitor of acetylcholine esterase in brain and is a leading therapeutic agent in the treatment of the Alzheimer's disease. Beyond its neuroprotective use, rivastigmine has also been employed for lowering intraocular pressure (see e.g., J Ocul Pharmacol Ther. 2000 February; 16(1):29-35). Furthermore, rivastigmine and galantamine were also reported to ameliorate or reduce presbyopia as disclosed in US 2011/0152274. While galantamine hydrobromide (Reminyl) is generally used in the treatment of Alzheimer's patients with mild to moderate cognitive deficits, galantamine when applied topically on the cornea, has also been shown to reduce intraocular pressure, as disclosed in WO 2007/016793.

Therefore, while brimonidine, rivastigmine, and galantamine are disclosed for certain ophthalmic uses, all or almost all of them suffer from several drawbacks and have not been considered in the treatment of NVD. Likewise, while other known compounds have been used in a variety of ophthalmic formulations, these formulations were also not shown effective or had even been considered for their use in the acute and transient treatment of NVD. Therefore, there remains a need for improved compositions and methods for treatment of ophthalmic conditions, and especially NVD.

### SUMMARY OF THE INVENTION

The inventive subject matter is directed to various compositions and methods of use of brimonidine, and/or rivastigmine, and/or galantamine at low concentrations to provide acute and transient treatment of NVD. Most typically, such compositions are topically applied and achieve a miotic effect sufficient to reduce one or more symptoms of NVD with a duration of between about 1-4 hours. It should be recognized, however, that in most (if not all) cases, the miotic effect is not sufficient to treat myopia.

In one aspect of the inventive subject matter, the inventors contemplate topical ophthalmic compositions for acute and transient treatment of night vision disturbance, and methods of acute and transient treatment of night vision disturbance in an individual. Most typically, methods will include a step of topically administering an ophthalmic formulation to an eye of individual, wherein the ophthalmic formulation comprises brimonidine in an amount of equal or less than 0.1%, and/or rivastigmine in an amount of equal or less than 1.0%, and/or galantamine in an amount of equal or less than 2.0%. Therefore, the inventors also contemplate topical ophthalmic compositions comprising brimonidine in an amount of equal or less than 0.1%, and/or rivastigmine in an amount of equal or less than 1.0%, and/or galantamine in an amount of equal or less than 2.0%.

In some embodiments, the ophthalmic formulation comprises brimonidine, but not rivastigmine or galantamine, in other embodiments the ophthalmic formulation comprises rivastigmine, but not brimonidine or galantamine, while in still other embodiments the ophthalmic formulation comprises galantamine, but not brimonidine or rivastigmine. Yet further contemplated embodiments include those in which the ophthalmic formulation comprises brimonidine and rivastigmine, but not galantamine, in which the ophthalmic formulation comprises brimonidine and galantamine, but not rivastigmine, and in which the ophthalmic formulation comprises brimonidine, rivastigmine, and galantamine.

For example, brimonidine, if present in the ophthalmic formulation, may be present in an amount of between 0.01% to 0.1% or in an amount of equal or less than 0.05%. Rivastigmine, if present in the ophthalmic formulation, may be present in an amount of between 0.1% to 1.0% or in an amount of between 0.5% to 1.0%. Galantamine, if present in the ophthalmic formulation, may present in an amount of between 0.2% to 2.0%, or in an amount of between 0.5% to 1.5%. Where desired, the ophthalmic formulation may further comprise carbachol, typically in an amount of equal or less than 1.5%, or in an amount of equal or less than 0.75%, or in an amount of equal or less than 0.15%.

It is further contemplated that the acute and transient treatment may results in an at least 10% (or at last 15%, or at least 20%) reduction in pupillary diameter that last for between 1 hour and no more than 6 hours, or that may last between 1 hour and no more than 4 hours, or that may last for no more than 4 or no more than 1 hour. Preferably, wherein the treatment reduces one or more than one of glare, starburst, halo, and double vision. As further needed or desired, contemplated ophthalmic formulations may also include benzalkonium chloride, preferably at an amount that increases delivery of brimonidine, rivastigmine, galantamine, and/or carbachol to the pupillary muscle. For example, suitable amounts of benzalkonium chloride in the ophthalmic formulation may be at least 0.001% but no more than 0.02%. As will be readily appreciated, the ophthalmic formulation is typically sterile and may be filled into a single-use container or multi-dose container.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a graph depicting strength and extended duration of the miotic effect of phentolamine in a rabbit eye model.

FIG. 2 is a graph depicting strength and duration of the miotic effect of brimonidine in a rabbit eye model.

FIGS. 3A and 3B are graphs depicting strength and duration of the miotic effect of rivastigmine alone (3A) or rivastigmine in combination with brimonidine (3B) in a rabbit eye model.

FIGS. 4A and 4B are graphs depicting strength and duration of the miotic effect of galantamine alone (4A) or galantamine in combination with brimonidine (34B) in a rabbit eye model.

FIG. 5 is a graph depicting strength and duration of the miotic effect of carbachol in a rabbit eye model.

FIGS. 6A-6C are graphs depicting the effect of benzalkonium chloride on carbachol effect at varying concentrations of benzalkonium chloride.

### DETAILED DESCRIPTION

Acute and transient treatment of NVD requires a sufficiently high concentration of a miotic agent to achieve a therapeutically effective miotic effect, but at the same time requires a relatively short duration of the miotic effect as NVD is in most cases limited to scotopic conditions. Unfortunately, the time for the pupil to recover to half of maximal constriction (t) is commonly estimated to be 7-8 hours for various miotic drugs (see e.g., Investigative Ophthalmology 1966). Viewed from a different perspective, the strength and the duration of the miotic effect tend to be correlated. In practice, NVD only requires correction during low light conditions such as an evening or night commute and should not subsequently affect vision after low light conditions have ceased, and as such long-term miosis (e.g., 7-15 hours) is undesirable. Therefore, compositions and methods are desired that provide an acute and transient miotic effect that is sufficiently strong to treat NVD but sufficiently limited in time.

The inventors have now discovered various compositions and methods for brimonidine, rivastigmine, and/or galantamine that allow for acute and transient treatment of NVD. In especially preferred aspects, the compositions are topically applied ophthalmic formulations that include brimonidine and/or rivastigmine and/or galantamine, and optionally carbachol, as the principal active pharmaceutical agent. The principal active pharmaceutical agent is present in the ophthalmic formulation at a rather low concentration of, for example, equal or less than 2.0% for galantamine, or equal or less than 1.0% rivastigmine, or equal or less than 0.1% for brimonidine. Unexpectedly, the inventors discovered that such low concentrations afforded a significant therapeutic effect with regard to NVD while having a controlled limited therapeutic duration, typically between 1-4 hours.

Brimonidine is an alpha adrenergic agonist. The chemical name for brimonidine is 5-Bromo-6-(2-imidazolidinylideneamino) quinoxaline L-tartrate. Brimonidine is available from various sources, including Allergan Inc, Irvine, CA. The tartrate salt of brimonidine is represented by the following formula:

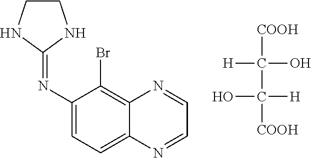

Rivastigmine is represented by the following formula, wherein ‘*’ denotes the presence of a chiral center. The chemical name for Rivastigmine is N-ethyl-3-[1-dimethyl amino)ethyl]-N-methylphenyl carbamate.

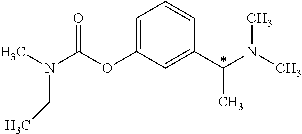

The molecular structure of galantamine is represented by the following formula:

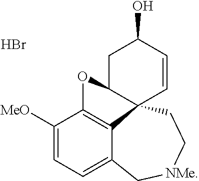 [SMILES: Br ; COc1ccc2c3c1O[C@H]1C[C@@H](O)C=C[C@@]31CCN(C)C2]

It should further be appreciated that contemplated and preferred compositions and methods are intended to treat NVD, which are generally associated with visual disturbances at longer distances (i.e., focal distances well beyond reading distances typically seen in treatment of presbyopia), typically distances of at least 1 m, or at least 5 m, or at least 10 m, or at least 25 m, or at least 50 m, and significantly longer distances. NVD symptoms typically include one or more of glare, starburst, halo, and double vision that are experienced under scotopic conditions. Therefore, particularly contemplated and preferred compositions and methods are intended to reduce or prevent one or more NVD symptoms in low-light environments such as evening and night. Thus, it should be noted that contemplated compositions and methods treat NVD without (substantially) affecting overall brightness. In addition, it should be appreciated that NVD as contemplated herein need not only be due to refractive surgery, but may have various other etiologies, including ageing, cataracts, excessive sunlight exposure, retinitis pigmentosa, vitamin A and/or zinc deficiency, etc.

Therefore, compositions and methods contemplated herein will typically effect a reduction of the pupillary diameter of about 10-30% (e.g., about 10% or about 15% or about 20% or about 25% reduction), which would be unsuitable or even entirely ineffective in a treatment of presbyopia. Interestingly, such reduction in pupillary size (particularly under low light conditions), will typically cover most of the area treated in surgical corneal correction such as LASIK procedures.

For example, contemplated formulations of the inventive subject matter can be advantageously provided in a ready-to-use format, preferably in form of an multi-dose eye dropper container or single use eye drop BFS (blow-fill-seal) package, which will eliminate microbial contamination risks. Most typically, contemplated formulations will be available in a range of concentrations commonly required by medical practitioners for treatment of NVD, and particularly acute and transient treatment of NVD.

In one embodiment, brimonidine is present in formulations in an amount of equal or less than 1.0 wt %, or in an amount of equal or less than 0.5 wt %, or in an amount of equal or less than 0.2 wt %, or in an amount of equal or less than 0.15 wt %. For example, the brimonidine may be present in the ophthalmic composition in an amount of between 0.01% and 0.05 wt %, between 0.02 wt % and 0.05 wt %, or between 0.05 wt % and 0.075 wt %, or between 0.075 wt % and 0.1 wt %, or between 0.1 wt % and 0.15 wt %, or between 0.15 wt % and 0.175 wt %, or between 0.175 wt % and 0.2 wt %. As will be readily appreciated, brimonidine for the preparation of contemplated formulations may be brimonidine or any suitable pharmaceutically acceptable salt thereof, including mineral salts (e.g., HCl salt), organic salts (e.g., sulfate), and tartrates. Similarly, where desired, the brimonidine may also be used in any suitable prodrug form.

For example, in one exemplary embodiment, the concentration of brimonidine in contemplated compositions is from about 0.015% to about 0.035% (w/w); or from about 0.035% to about 0.055% (w/w), or from about 0.055% to about 0.075% (w/w), or from about 0.075% to about 0.095% (w/w), or from about 0.095% to about 0.115% (w/w), or from about 0.115% to about 0.135% (w/w), or from about 0.135% to about 0.155% (w/w) or from about 0.155% to about 0.175% (w/w), or from about 0.175% to about 0.195% (w/w) or from about 0.195% to about 0.215% (w/w).

In another exemplary embodiment, the concentration of brimonidine in contemplated in formulations is from about 0.025% to about 0.050% (w/w); or from about 0.050% to about 0.075% (w/w), or from about 0.075% to about 0.1% (w/w), or from about 0.1% to about 0.15% (w/w), or from about 0.15% to about 0.25% (w/w), or from about 0.25% to about 0.50% (w/w), or from about 0.50% to about 0.75% (w/w) or from about 0.75% to about 1.00% (w/w).

In still further exemplary embodiments, the concentration of brimonidine in contemplated formulations is about 0.01%, or about 0.02%, or about 0.03%, or about 0.04%, or about 0.05%, or about 0.06%, or about 0.07%, or about 0.08%, or about 0.09%, or about 0.1%, or about 0.025%, or about 0.035%, or about 0.045%, or about 0.055%, or about 0.065%, or about 0.075%, or about 0.085%, or about 0.095%, or about 0.15%, or about 0.20%, or about 0.25%, or about 0.30%, or about 0.35%, or about 0.40%, or about 0.45%, or about 0.50%, or about 0.55%, or about 0.60%, or about 0.65%, or about 0.70%, or about 0.75%, or about 0.80%, or about 0.85%, or about 0.90%, or about 0.095%, or about 0.10%, or about 0.11%, or about 0.11%, or about 0.12%, or about 0.12%, or about 0.13%, or about 0.13%, or about 0.14%, or about 0.14%, or about 0.15%, or about 0.15%, or about 0.17%, or about 0.17%, or about 0.19%, or about 0.20% (w/w).

In another embodiment, rivastigmine is typically present in formulations in an amount of equal or less than 5 wt %, in an amount of equal or less than 2.5 wt %, in an amount of equal or less than 1 wt %, or in an amount of equal or less than 0.5 wt %, or in an amount of equal or less than 0.1 wt %. For example, the rivastigmine may be present in the ophthalmic composition in an amount of between 0.001% and 0.01 wt %, between 0.01 wt % and 0.1 wt %, or between 0.1 wt % and 1.0 wt %. As will be readily appreciated, rivastigmine for the preparation of contemplated formulations may be rivastigmine or any suitable pharmaceutically acceptable salt thereof, including mineral salts (e.g., HCl salt) and organic salts (e.g., sulfate). Similarly, where desired, the rivastigmine may also be used in any suitable prodrug form.

For example, in one exemplary embodiment, the concentration of rivastigmine in contemplated formulations is from about 0.015% to about 0.05% (w/w); or from about 0.05% to about 0.1% (w/w), or from about 0.1% to about 0.15% (w/w), or from about 0.15% to about 0.2% (w/w), or from about 0.2% to about 0.3% (w/w), or from about 0.3% to about 0.4% (w/w), or from about 0.4% to about 0.5% (w/w) or from about 0.5% to about 0.6% (w/w), or from about 0.6% to about 0.7% (w/w), or from about 0.7% to about 0.8% (w/w), or from about 0.8% to about 0.9% (w/w) or from about 0.9% to about 1.0% (w/w), or from about 1.0% to about 1.2% (w/w), or from about 1.2% to about 1.5% (w/w), or from about 1.5% to about 1.7% (w/w), or from about 1.7% to about 2.0% (w/w), and even higher.

In another exemplary embodiment, the concentration of rivastigmine in contemplated formulations is from about 0.25% to about 0.50% (w/w); or from about 0.50% to about 0.75% (w/w), or from about 0.75% to about 1.0% (w/w), or from about 1.0% to about 1.25% (w/w), or from about 1.25% to about 1.5% (w/w), or from about 1.5% to about 1.75% (w/w), or from about 1.75% to about 2.0% (w/w).

In still further exemplary embodiments, the concentration of rivastigmine in contemplated formulations is about 0.001%, or about 0.005%, or about 0.01%, or about 0.05%, or about 0.1%, or about 0.2%, or about 0.3%, or about 0.4%, or about 0.5%, or about 0.6%, or about 0.7%, or about 0.8%, or about 0.9%, or about 1.0%, or about 1.2%, or about 1.4%, or about 1.6%, or about 1.8%, or about 2.0%.

In yet another embodiment, galantamine is present in the formulations in an amount of equal or less than 5.0 wt %, in an amount of equal or less than 4.0 wt %, in an amount of equal or less than 3.0 wt %, in an amount of equal or less than 2.5 wt %, or in an amount of equal or less than 0.2 wt %, or in an amount of equal or less than 0.1 wt %. For example, the galantamine may be present in the ophthalmic composition in an amount of between 0.01% and 0.1 wt %, between 0.05 wt % and 0.5 wt %, or between 0.2 wt % and 2.0 wt %. As will be readily appreciated, galantamine for the preparation of contemplated formulations may be galantamine or any suitable pharmaceutically acceptable salt thereof, including mineral salts (e.g., HCl salt) and organic salts (e.g., sulfate). Similarly, where desired, the galantamine may also be used in any suitable prodrug form.

For example, in one exemplary embodiment, the concentration of galantamine in contemplated formulations is from about 0.005% to about 0.01% (w/w), or from about 0.01% to about 0.05% (w/w), or from about 0.05% to about 0.1% (w/w), or from about 0.1% to about 0.25% (w/w), or from about 0.25% to about 0.5% (w/w), or from about 0.5% to about 0.75% (w/w) or from about 0.75% to about 1.0% (w/w), or from about 0.5% to about 0.75% (w/w) or from about 0.75% to about 1.0% (w/w), or from about 1.0% to about 1.5% (w/w) or from about 1.5% to about 2.0% (w/w).

In another exemplary embodiment, the concentration of galantamine in contemplated formulations is from about 0.05% to about 0.1% (w/w); or from about 0.1% to about 0.15% (w/w), or from about 0.15% to about 0.3% (w/w), or from about 0.3% to about 0.5% (w/w), or from about 0.5% to about 0.75% (w/w), or from about 0.75% to about 1.0% (w/w), or from about 1.0% to about 1.5% (w/w) or from about 1.5% to about 2.0% (w/w), or from about 2.0% to about 2.5% (w/w), or from about 2.5% to about 3.0% (w/w).

In still further exemplary embodiments, the concentration of galantamine in contemplated formulations is or about 0.05%, or about 0.1%, or about 0.1%, or about 0.25%, or about 0.35%, or about 0.45%, or about 0.55%, or about 0.65%, or about 0.75%, or about 0.85%, or about 0.95%, or about 1.0%, or about 1.1%, or about 1.2%, or about 1.3%, or about 1.4%, or about 1.5%, or about 1.6%, or about 1.7%, or about 1.8%, or about 1.9%, or about 2.0% (w/w).

Throughout this disclosure, it should be noted that all possible permutations and combinations of brimonidine, rivastigmine, and galantamine are contemplated in the disclosed ophthalmic compositions, typically at the concentrations noted above. For example, the ophthalmic compositions disclosed herein may comprise brimonidine but not rivastigmine or galantamine. In another embodiment, the ophthalmic composition may comprise rivastigmine but not brimonidine or galantamine. In another embodiment, the ophthalmic composition may comprise galantamine but not brimonidine or rivastigmine. In another embodiment, the ophthalmic composition may comprise brimonidine and rivastigmine but not galantamine. In another embodiment, the ophthalmic composition may comprise brimonidine and galantamine but not rivastigmine. In another embodiment, the ophthalmic composition may comprise brimonidine, rivastigmine, and galantamine.

In still further contemplated compositions, carbachol may be present in the ophthalmic formulations contemplated herein. For example, carbachol may be present in formulations in an amount of equal or less than 0.5 wt %, or in an amount of equal or less than 0.2 wt %, or in an amount of equal or less than 0.15 wt %. For example, the carbachol may be present in the ophthalmic composition in an amount of between 0.01% and 0.05 wt %, between 0.02 wt % and 0.05 wt %, or between 0.05 wt % and 0.075 wt %. As will be readily appreciated, carbachol for the preparation of contemplated formulations may be carbachol or any suitable pharmaceutically acceptable salt thereof, including mineral salts (e.g., HCl salt) and organic salts (e.g., sulfate). Similarly, where desired, the carbachol may also be used in any suitable prodrug form.

For example, in one exemplary embodiment, the concentration of carbachol in contemplated carbachol formulations is from about 0.015% to about 0.025% (w/w); or from about 0.025% to about 0.035% (w/w), or from about 0.035% to about 0.045% (w/w), or from about 0.045% to about 0.055% (w/w), or from about 0.055% to about 0.065% (w/w), or from about 0.065% to about 0.75% (w/w), or from about 0.075% to about 0.085% (w/w) or from about 0.085% to about 0.1% (w/w).

In another exemplary embodiment, the concentration of carbachol in contemplated carbachol formulations is from about 0.025% to about 0.050% (w/w); or from about 0.050% to about 0.075% (w/w), or from about 0.075% to about 0.1% (w/w), or from about 0.1% to about 0.15% (w/w), or from about 0.15% to about 0.25% (w/w), or from about 0.25% to about 0.50% (w/w), or from about 0.50% to about 0.75% (w/w) or from about 0.75% to about 1.00% (w/w).

In still further exemplary embodiments, the concentration of carbachol in contemplated carbachol formulations is about 0.01%, or about 0.02%, or about 0.03%, or about 0.04%, or about 0.05%, or about 0.06%, or about 0.07%, or about 0.08%, or about 0.09%, or about 0.1%, or about 0.025%, or about 0.035%, or about 0.045%, or about 0.055%, or about 0.065%, or about 0.075%, or about 0.085%, or about 0.095%, or about 0.15%, or about 0.20%, or about 0.25%, or about 0.30%, or about 0.35%, or about 0.40%, or about 0.45%, or about 0.50%, or about 0.55%, or about 0.60%, or about 0.65%, or about 0.70%, or about 0.75%, or about 0.80%, or about 0.85%, or about 0.90%, or about 0.095%, or about 0.10%, or about 0.11%, or about 0.11%, or about 0.12%, or about 0.12%, or about 0.13%, or about 0.13%, or about 0.14%, or about 0.14%, or about 0.15%, or about 0.15%, or about 0.17%, or about 0.17%, or about 0.19%, or about 0.20% (w/w).

As will be readily appreciated, contemplated compositions will be in most cases aqueous compositions (i.e., use water as single solvent). However, it is noted that non-aqueous solvents are also deemed suitable as co-solvents, and particularly suitable co-solvents include glycols, alcohols, polyols, etc. Viewed from another perspective, where a co-solvent is employed, especially preferred cosolvents are miscible with water in an amount of at least 10% and will not separate into a distinct phase.

It is further contemplated that the compositions presented herein will comprise a buffer, and suitable buffers are generally buffers that stabilize the pH of the contemplated liquid formulations in a near-neutral pH range, for example between pH 4.0 and 9.0, or between pH 4.5 and 8.0, and more preferably between pH 6.0 and 7.5. Therefore, and most typically the pH of contemplated formulations will be equal or less than 8.0 and more typically equal or less than 7.5, and most typically less than 7.0, but higher than 4.5, more typically higher than 5.0, and most typically higher than 5.2. For example, suitable carbachol compositions may have a pH of 5.0 (+/−0.2), or a pH of 5.5 (+/−0.2), or a pH of 6.0 (+/−0.2), or a pH of 6.5 (+/−0.2), or a pH of 7.0 (+/−0.2), or a pH of 7.5 (+/−0.2).

Most typically, the buffer system and/or buffer may have a buffer strength that is relatively low, for example, equal or less than 100 mM, equal or less than 75 mM, equal or less than 60 mM, equal or less than 50 mM, or between 5 mM and 50 mM (e.g., 10 mM, 20 mM, 30 mM, 40 mM). Therefore, in exemplary embodiments, the buffering system is in the pharmaceutical composition in a concentration of from about 10 mM to about 75 mM, or from about 10 mM to about 60 mM, or from about 0.1 mM to about 60 mM, or from about 0.1 mM to about 55 mM, or from about 0.1 mM to about 50 mM, or from about 5 mM to about 60 mM, or from about 0.1 mM to about 10 mM, or from about 1 mM to about 10 mM, or from about 9 mM to about 20 mM, or from about 15 mM to about 25 mM, or from about 19 mM to about 29 mM, or from about 24 mM to about 34 mM, or from about 29 mM to about 39 mM, or from about 34 mM to about 44 mM, or from about 39 mM to about 49 mM, or from about 44 mM to about 54 mM, or from about 19 mM to about 54 mM, or from about 25 mM to about 54 mM.

Of course, it should be appreciated that there are many types of buffer systems and buffers known in the art, and all of those are deemed suitable for use herein, including buffer systems comprising an acid and a salt of the acid, a first and a second salt (e.g., monobasic and dibasic salt), and amphoteric buffer molecules. For example, suitable buffer systems with an acid and a salt of the acid include citric acid/sodium citrate buffers, ethanoic acid/sodium ethanoate buffers, boric acid/sodium borate, while suitable buffers having a first and a second salt include monobasic sodium phosphate/dibasic sodium phosphate, or monobasic sodium phosphate/sodium citrate, etc. Similarly, suitable amphoteric buffer molecules include HEPES, MOPS, PIPES, MES, etc.

Where desired, the formulation may also include one or more chelating agents, and particularly metal ion chelators. For example, suitable chelators include various bicarboxylic acids, tricarboxylic acids, and aminopolycarboxylic acids such as ethylenediaminetetraacetic acid (EDTA), ethylene glycol-bis(β-aminoethyl ether)-N,N,N′,N′-tetraacetic acid (EGTA), and penta(carboxymethyl)diethylenetriamine (DTPA), and salts and hydrates thereof. For example, exemplary chelator concentrations are between 10 μg/ml and 50 μg/ml, between 50 μg/ml and 250 μg/ml, and between 100 μg/ml and 500 μg/ml. Viewed form a different perspective, chelator concentrations of equal or less than 0.03 wt %, or equal or less than 0.02 wt %, or equal or less than 0.01 wt % are especially advantageous.

Suitable chelating agents include monomeric polyacids such as EDTA, cyclohexanediamine tetraacetic acid (CDTA), hydroxyethylethylenediamine triacetic acid (HEDTA), diethylenetriamine pentaacetic acid (DTPA), dimercaptopropane sulfonic acid (DMPS), dimercaptosuccmic acid (DMSA), aminotrimethylene phosphonic acid (ATPA), citric acid, ophthalmologically acceptable salts thereof, and combinations of any of the foregoing. Further suitable chelating agents include pyrophosphates, tripolyphosphates, and, hexametaphosphates, chelating antibiotics such as chloroquine and tetracycline, nitrogen-containing chelating agent containing two or more chelating nitrogen atoms within an imino group or in an aromatic ring (e.g., diimines, 2,2′-bipyridines, etc.), and various polyamines such as cyclam (1,4,7,11-tetraazacyclotetradecane), N—(C1-C30 alkyl)-substituted cyclams (e.g., hexadecyclam, tetramethylhexadecylcyclam), diethylenetriamine (DETA), spermine, diethylnorspermine (DENSPM), diethylhomo-spermine (DEHOP), and deferoxamine (N′-[5-[[4-[[5-(acetylhydroxyamino)pentyl]amino]-1,4-dioxobutyl]hydroxy-amino]pentyl]-N′-(5-aminopentyl)-N-hydroxybutanediamide; also known as desferrioxamine B and DFO).

In still further contemplated aspects, the formulations may also include a salt as a tonicity agent. With respect to suitable salts it is contemplated that the salt is a pharmaceutically acceptable salt, and especially NaCl, at a concentration of at least 0.2 wt %, or at least 0.4 wt %, or at least 0.5 wt %, or at least 0.7 wt %. For example, suitable salt concentrations are between 0.2 wt % and 1.1 wt %, 0.4 wt % and 0.9 wt %, or 0.3 wt % and 0.7 wt %. Depending on the particular salt concentration, additional tonicity agents may be added, and suitable tonicity agents include glycerol, thioglycerol, mannitol, lactose, and dextrose. The amount of tonicity adjusting agent used can be adjusted to obtain osmolality of the formulations in the range of 260 to 340 mOsm/kg. An osmometer can be used to check and adjust the amount of tonicity adjusting agent to be added to obtain the desired osmolality.

As contemplated formulations are used as an ophthalmic formulation, it is generally preferred that the formulation also includes a viscosity modifier to adjust the viscosity of the formulation to a dynamic viscosity of between 5 and 50 cP (centipoise), and more preferably between 10 and 40 cP, and most preferably between 10 to 30 cP. While there are numerous viscosity modifiers known in the art such as various polymers, glycerol, and polysaccharidic polymers (all of which are contemplated herein), especially preferred viscosity modifiers include cellulosic viscosity modifiers. For example, particularly preferred cellulosic viscosity modifiers include modified and unmodified hydroxyethyl cellulose, hydroxypropyl cellulose, and hydroxypropyl methylcellulose.

As will be readily appreciated, the exact quantity of the viscosity modifier may vary depending on the type of modifier used and desired final viscosity. For example, where the viscosity modifier is a cellulosic modifier and the final viscosity should be between 1 and 30 cP, suitable quantities of the modifier will typically be in the range of 0.5 wt % (+/−0.1 wt %) of the ophthalmic carbachol composition. The person of ordinary skill will be readily able to adjust the viscosity to a desired measure using viscometers (e.g., rotational, vibration, etc.) well known in the art.

In exemplary embodiments, suitable concentrations of the viscosity modifier in contemplated ophthalmic formulations may be any value less than 5% (w/w). For example, suitable concentrations of the viscosity modifier include 0.01% to 4.99% (w/w); or 0.05% to 4.50% (w/w), 0.10% to 3.50% (w/w), 0.15% to 3.00% (w/w), 0.20% to 2.50% (w/w), 0.21% to 2.20% (w/w), 0.22% to 2.10% (w/w), 0.23% to 2.00% (w/w), 0.24% to 1.90% (w/w); 0.25% to 1.80% (w/w), 0.26% to 1.70% (w/w), 0.27% to 1.60% (w/w), 0.28% to 1.50% (w/w), 0.29% to 1.40% (w/w), 0.30% to 1.30% (w/w), 0.31% to 1.2% (w/w), 0.32% to 1.10% (w/w), 0.33% to 1.00% (w/w), 0.34% to 0.90% (w/w); 0.35% to 0.80% (w/w), 0.36% to 0.75% (w/w), 0.37% to 0.70% (w/w), 0.38% to 0.69% (w/w), 0.39% to 0.68% (w/w), 0.40% to 0.67% (w/w), 0.41% to 0.66% (w/w), 0.42% to 0.65% (w/w), 0.43% to 0.64% (w/w), 0.44% to 0.63% (w/w), 0.45% to 0.62% (w/w), 0.45% to 0.61% (w/w), 0.45% to 0.60% (w/w), 0.45% to 0.59% (w/w), 0.45% to 0.58% (w/w), 0.45% to 0.57% (w/w), 0.45% to 0.56% (w/w), 0.45% to 0.55% (w/w), 0.46% to 0.54% (w/w), 0.47% to 0.53% (w/w), 0.48% to 0.52% (w/w) or 0.49% to 0.51% (w/w).

Therefore, appropriate concentrations of the viscosity modifier in contemplated ophthalmic formulations include 0.01%, 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.51%, 0.52%, 0.53%, 0.54%, 0.55%, 0.56%, 0.57%, 0.58%, 0.59%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99%, 1.00%, 1.10%, 1.20%, 1.30%, 1.40%, 1.50%, 1.60%, 1.70%, 1.80%, 1.90%, 2.00%, 2.10%, 2.20%, 2.30%, 2.40%, 2.50%, 2.60%, 2.70%, 2.80%, 2.90%, 3.00%, 3.10%, 3.20%, 3.30%, 3.40%, 3.50%, 3.60%, 3.70%, 3.80%, 3.90%, 4.00%, 4.10%, 4.20%, 4.30%, 4.40%, 4.50%, 4.60%, 4.70%, 4.80%, 4.90% and 4.99% (w/w).

As needed or desired, contemplated formulations will further include one or more preservatives such as benzalkonium chloride, cetrimide or cetrimonium chloride or bromide, benzododecinium bromide, miramine, cetylpyridinium chloride, polidronium chloride or polyquaternium-1, polyquaternium-42 (also known as polixetonium), sepazonium chloride; mercurial derivatives such as the phenylmercury salts (acetate, borate or nitrate), mercuriothiolate sodium (otherwise called thiomersal or thimerosal) and mercurobutol; amidines such as chlorhexidine digluconate or polyhexamethylene biguanide (PHMB); alcohols such as chlorobutanol or phenylethanol or benzyl alcohol or phenol or m-cresol or phenoxyethanol; parabens or esters such as parahydroxybenzoic acid, methylparaben, and propylparaben).

With respect to the sterilization of contemplated formulations it should be appreciated that contemplated formulations may be sterilized using all known manners of sterilization, including filtration through 0.22 micron filters, heat sterilization, autoclaving, and/or radiation (e.g., gamma, electron beam, microwave).

Viewed from a different perspective, it is therefore noted that the compositions according to the inventive subject matter are formulated such that the miotic effect will be achieved within a relatively short time (i.e., acute treatment) and will have a relatively short duration (i.e., transient treatment) while providing a therapeutic effect with respect to NVD (i.e., reduce or eliminate at least one of glare, halo, double vision, and starburst).

Most typically, acute treatment will result in a maximum pupillary constriction within no more than 60 min, or no more than 50 min, or no more than 40 min, or no more than 30 min, or no more than 20 min, or no more than 15 min, or no more than 10 min from administration of the ophthalmic composition. Thus, maximum pupillary constriction may be observed within 5-10 minutes, or within 10-20 minutes, or within 20-30 minutes, or within 30-40 minutes, or within 40-50 minutes, or within 50-60 minutes. With respect to transient treatment, it is contemplated that the duration of the miotic effect between administration and recovery to at 70% of initial pupillary diameter is at least 10 min, or at least 20 min, or at least 30 min, or at least 40 min, or at least 60 min, or at least 90 min, or at least 120 min, or at least 180 min, or at least 240 min, or at least 300 min, or at least 360 min, and less preferably at least 7 hours, or at least 8 hours, or even more in some cases (such duration can be determined using laser pupillometry in a rabbit eye test model). On the other hand it is generally preferred that the duration of the miotic effect between administration and recovery to at 70% of initial pupillary diameter is no longer than 8 hours, or no longer than 7 hours, and more typically no longer than 6 hours, or no longer than 5 hours, or no longer than 4.5 hours, or no longer than hours, or no longer than 3.5 hours, or no longer than 3 hours, or no longer than 2.5 hours, or no longer than 2 hours. Thus, the duration of the miotic effect between administration and recovery to at 70% of initial pupillary diameter may last between 60-90 minutes, or between 90 and 120 minutes, or between 120-180 minutes, or between 2-3 hours, or between 3-4 hours, or between 4-5 hours.

Most typically, the miotic effect produced by contemplated compositions will be produce a reduction in pupillary diameter (as measured from before administration) of at least 5%, or at least 7%, or at least 10%, or at least 12%, or at least 14%, or at least 16%, or at least 18%, or at least 20%, or at least 22%, or at least 25%, or at least 30%, or at least 35%, or at least 40%, or at least 50%, but most typically no more than 30%, or no more than 25%, or no more than 22%, or no more than 20%, or no more than 18%, or no more than 15%. Therefore, contemplated reductions in pupillary diameter (as measured from before administration) will be between 2-5%, or between 5-7%, or between 7-10%, or between 10-12%, or between 12-15%, or between 15-17%, or between 17-20%, or in some cases even between 20-25% or even higher.

Notably, and as shown in more detail further below, the inventors also discovered that benzalkonium chloride may enhance the miotic effect. While not wishing to be bound by any theory or hypothesis, benzalkonium chloride is thought to assist brimonidine, rivastigmine, galantamine, and/or carbachol to penetrate to the pupillary muscle, and as such the effective concentration of benzalkonium chloride will be higher. Most typically, concentrations of benzalkonium chloride will be higher than those normally used for antimicrobial activity.

For example, where benzalkonium chloride (BAC; N-Alkyl-N-benzyl-N,N-dimethyl-ammonium chloride) or other quaternary ammonium-based surfactant is being used to enhance carbachol penetration to the pupillary muscle, it should be noted that the concentration of the BAC will typically be above the concentration ordinarily used for antimicrobial effect. Most preferably, the concentration of BAC or other quaternary ammonium-based surfactant in contemplated formulations will be at least 0.010%, or at least 0.012%, or at least 0.014%, or at least 0.016%, or at least 0.018%, or at least 0.020%, or at least 0.022%, or at least 0.024%, or at least 0.026%, or at least 0.028%, or at least 0.030%, or at least 0.033%, or at least 0.036%, or at least 0.039%, or at least 0.042%, or at least 0.045%, or at least 0.050%, or even higher. Therefore, suitable BAC or other quaternary ammonium-based surfactant concentrations may be in the range of between 0.010-0.014%, or between 0.012-0.016%, or between 0.014-0.018%, or between 0.016-0.020%, or between 0.018-0.022%, or between 0.020-0.024%, or between 0.022-0.026%, or between 0.024-0.028%, or between 0.026-0.030%, or between 0.028-0.032%, or between 0.030-0.035%, or between 0.015-0.025%, or between 0.010-0.030%, or between 0.015-0.035%. Thus, and viewed form a different perspective, the concentration of BAC or other quaternary ammonium-based surfactant in contemplated formulations will be at least 0.015%, or at least 0.017%, or at least 0.0192%, or at least 0.017%, or at least 0.021%, or at least 0.023%, or at least 0.025%, or at least 0.030%, or even higher. In still further contemplated aspects, the concentration of the BAC or other quaternary ammonium-based surfactant may also be significantly lower, such as for example, about 0.001%, or about 0.002%, or about 0.003%, or about 0.004%, or about 0.005%, or higher.

Embodiments of the present disclosure are further described in the following examples. The examples are merely illustrative and do not in any way limit the scope of the invention as claimed.

### Examples

At least theoretically, there are multiple classes of drugs available that could be possibly used to induce miosis, including alpha-1 agonists (e.g., dapiprazole, doxazosin) as well as alpha-2 agonists (e.g., brimonidine) as both agonists act on the iris dilator and so could inhibit pupil dilation. On the other hand, cholinergic agonists (e.g., carbachol, pilocarpine, aceclidine) generally act on the iris sphincter and could so increase pupil constrictions. In a similar manner, it can be theorized that cholinesterase inhibitors (e.g., rivastigmine, galantamine, tacrine, neostigmine) can act on the iris sphincter and so increase pupil constrictions. However, despite the above functional characterization, none of these agents have been tested for use in treatment of NVD.

Moreover, and despite their specific mode of action, not all classes and even compounds of the same class act equally well in the context of acute and transient treatment of NVD as shown in more detail below. Notably, some of the drugs only had desirable effect only at relatively low concentration, and then for a desirably short duration, whereas other drugs had a significant and extended duration of miotic effect.

The following examples were performed to determine which active pharmaceutical agent (API), alone or in combination, could effect upon topical administration an approximate 1-2 mm decrease in pupillary diameter with a desirable (e.g., about 4 h) duration of action. Unless stated otherwise, a rabbit animal model was used for the studies. Phentolamine was used as a positive control (comparator API) and caused an approximately 1 mm decrease in the pupil at 0.5% and 1% with a duration of more than 6 hours. The inventors also found that carbachol was very potent, even at the lowest dose with a duration of action within a desirable relatively short time frame of 2-4 h. Moreover, the inventors found that brimonidine had little to no effect at higher doses tested and produced some miotic effect at doses at and below 0.1%. On the other hand, rivastigmine at a concentration of about 1% provided similar desirable results as carbachol. Notably, the addition of brimonidine did not improve the efficacy of rivastigmine. Likewise, galantamine at a dose of about 1-2% provided similar desirable results as carbachol. Once more, addition of brimonidine did not improve the efficacy of galantamine.

Study design: 3 Dutch Belted Rabbits (pigmented breed)/group received a single ocular administration of test articles (one drop per eye) at 3 concentrations (tested one week apart). After a 2-week rest period, combinations were tested. Ocular irritation was assessed using a modified Draize test. Intraocular pressure (IOP) was measured at baseline, 1 h, 4 h, 6 h and 24 hours. Pupillary Diameter was measured at baseline, 15 min, 1 h, 4 h and 6 h after dosing. Due to the variation in pupillary diameter and IOP between rabbits and between eyes within rabbits, the data for pupillary diameter and IOP were normalized and presented with error bars (SD). A change of 15% to 20% approximates a pupillary diameter change of 1 mm.

| | | | | | |
|---|---|---|---|---|---|
| Group | No of | | | | |
| ID | Animals | Treatment | Timepoint | Vol/Route/Dose | Endpoints |

Phase 1: Low Dose

| | | | | | |
|---|---|---|---|---|---|
| 1 | 3 | Brimonidine | Day 0 | Topical | Ocular examinations: Baseline |
| 2 | 3 | Rivastigmine | | drop per eye | (Day −1), 1, 4, 24 and 72 hrs |
| 3 | 3 | Galantamine | | Low | post-dose; |
| 4 | 3 | Carbachol | | | IOP: Baseline (Day −1), 1, 4, 6, |
| 5 | 3 | Phentolamine | | | 24, and 72 hrs post-dose; |
| | | | | | Pupillometry: Baseline, 15, 60, |
| | | | | | 240, and 360 minutes post- |
| | | | | | dose. |

Phase 2: Mid-level Dose

| | | | | | |
|---|---|---|---|---|---|
| 6 | 3 | Brimonidine | Day 7 | Topical | Ocular examinations: Baseline |
| 7 | 3 | Rivastigmine | | drop per eye | (Day −1), 1, 4, 24 and 72 hrs |
| 8 | 3 | Galantamine | | Middle | post-dose; |
| 9 | 3 | Carbachol | | | IOP: Baseline (Day −1), 1, 4, 6, |
| 10 | 3 | Phentolamine | | | 24, and 72 hrs post-dose; |
| | | | | | Pupillometry: Baseline, 15, 60, |
| | | | | | 240, and 360 minutes post- |
| | | | | | dose. |

Phase 3: High Dose

| | | | | | |
|---|---|---|---|---|---|
| 11 | 3 | Brimonidine | Day 14 | Topical | Ocular examinations: Baseline |
| 12 | 3 | Rivastigmine | | drop per eye | (Day −1), 1, 4, 24 and 72 hrs |
| 13 | 3 | Galantamine | | High | post-dose; |
| 14 | 3 | Carbachol | | | IOP: Baseline (Day −1), 1, 4, 6, |
| 15 | 3 | Phentolamine | | | 24, and 72 hrs post-dose; |
| | | | | | Pupillometry: Baseline, 15, 60, |
| | | | | | 240, and 360 minutes post- |
| | | | | | dose. |

The treatment schedule for various combination treatments with escalating doses of rivastigmine and galantamine are shown in the table below. One drop of the formulation was used in these experiments.

| Phase | Treatment | Endpoints |
|---|---|---|
| 5 | Brimonidine tartrate 0.1% + | Ocular Examination: |
| | Rivastigmine (0.01%) | Baseline (Day −1), 1, 4, |
| | Brimonidine tartrate 0.1% + | 24 and 72 h post-dose |
| | Galantamine Hydrobromide (0.02%) | IOP: |
| 6 | Brimonidine tartrate 0.1% + | Baseline (Day −1), 1, 4, |
| | Rivastigmine (0.1%) | 24 and 72 h post-dose |
| | Brimonidine tartrate 0.1% + | Pupillometry: |
| | Galantamine Hydrobromide (0.2%) | Baseline (Day −1), 1, 4, |
| 7 | Brimonidine tartrate 0.1% + | 24 and 72 h post-dose |
| | Rivastigmine (1%) | |
| | Brimonidine tartrate 0.1% + | |
| | Galantamine Hydrobromide (2%) | |

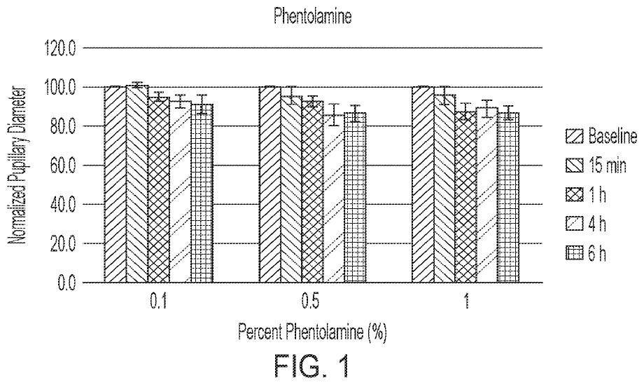

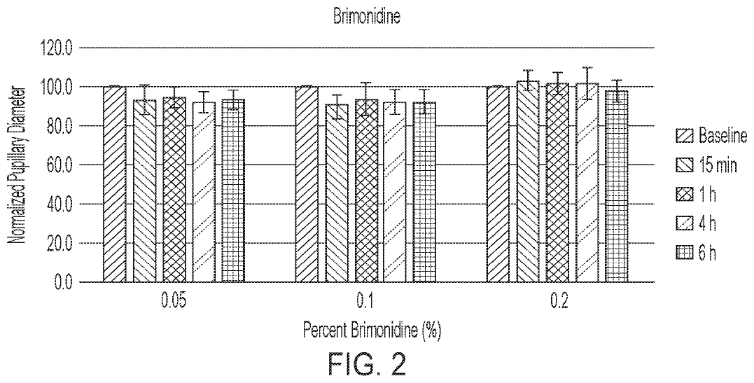

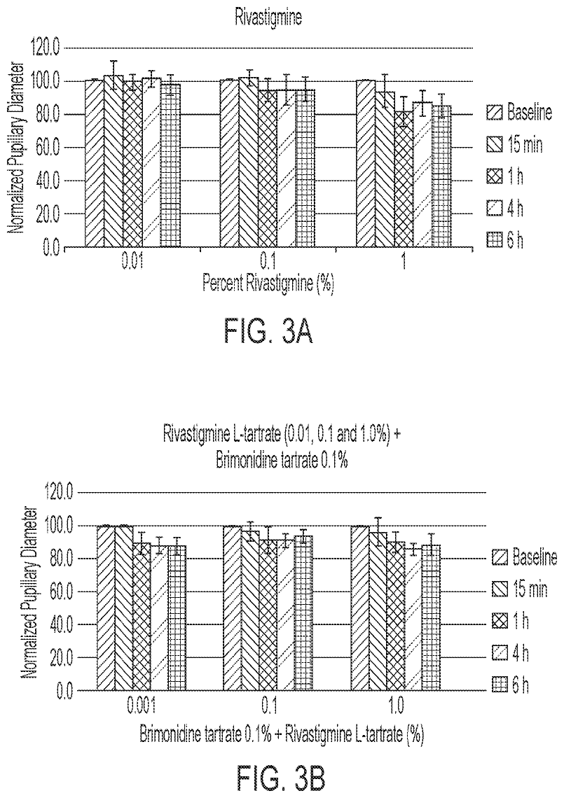

The results were grouped into data for three different concentrations of the API. More specifically, FIG. 1 shows the effect of topical ocular treatment with phentolamine on pupillary diameter in rabbits. As can be readily seen from the graphs, a reduction in pupillary diameter occurred at all doses and lasted for at least 6 h. FIG. 2 shows the effect of topical ocular treatment of brimonidine on pupillary diameter in rabbits. Here it is evident that brimonidine had little to no effect at doses at or above 0.2%, and moderate effect at doses at or below 0.1%. FIG. 3A depicts the effect of topical ocular treatment of rivastigmine alone and FIG. 3B depicts the effect of topical ocular treatment of rivastigmine in combination with brimonidine on pupillary diameter in rabbits. As is readily evident, rivastigmine 1% alone performed substantially similar to phentolamine (comparator). Interestingly, the addition of brimonidine had no detectable effect.

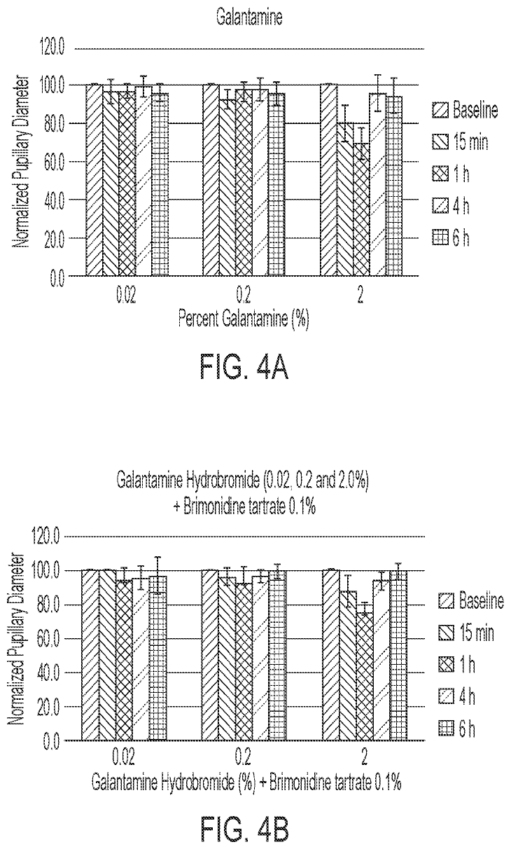

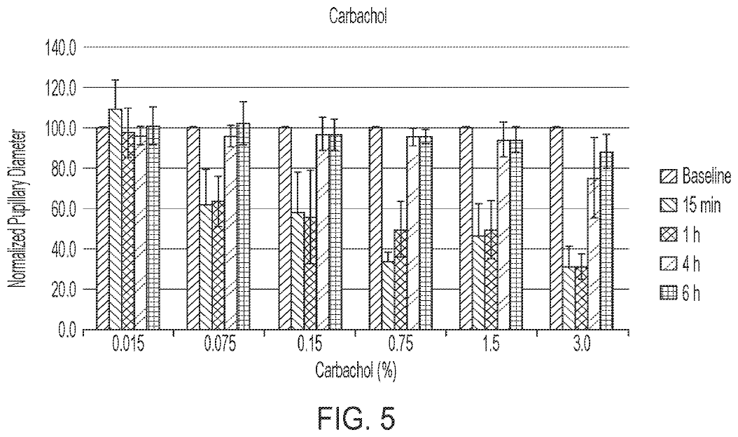

Similarly, FIG. 4A shows the effect of topical ocular treatment of galantamine alone and FIG. 4B shows the effect of topical ocular treatment of galantamine in combination with brimonidine on pupillary diameter in rabbits. As can be taken from the graphs, a reduction in pupillary diameter occurred at the high dose (2%) and lasted for at least 1-3 h, and brimonidine once more did not add to the miotic effect. FIG. 5 depicts the effect of carbachol on pupillary diameter in rabbits. As can be readily appreciated from the graph in FIG. 5, carbachol was very potent, even at a low concentration of about 0.075% in the rabbit eye model. Advantageously, the duration of action was relatively short, at between about 1-3 hours.

Phentolamine, the comparator, caused an approximately 1 mm decrease in the pupil at 0.5% and 1% with a duration of at least 6 h, and carbachol was very potent, even at low doses of about 0.075%. Notably, Brimonidine had little to no effect at doses tested, while Rivastigmine at a concentration of about 1% alone had desirable effect (1 mm decrease lasting at least 6 h) and Galantamine at a concentration of about 2% alone had desirable effect (1-2 mm decrease lasting at least 1 h). Brimonidine did not improve the efficacy of Rivastigmine or Galantamine. Beneficially, treatment with the tested compounds did not affect the IOP (minimal changes), nor cause ocular irritation.

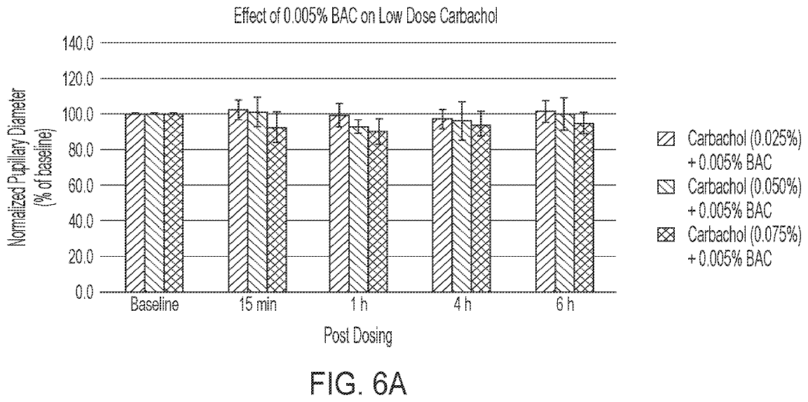

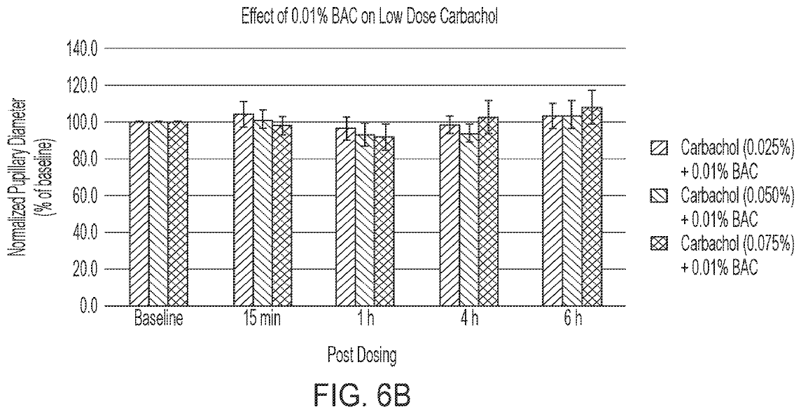

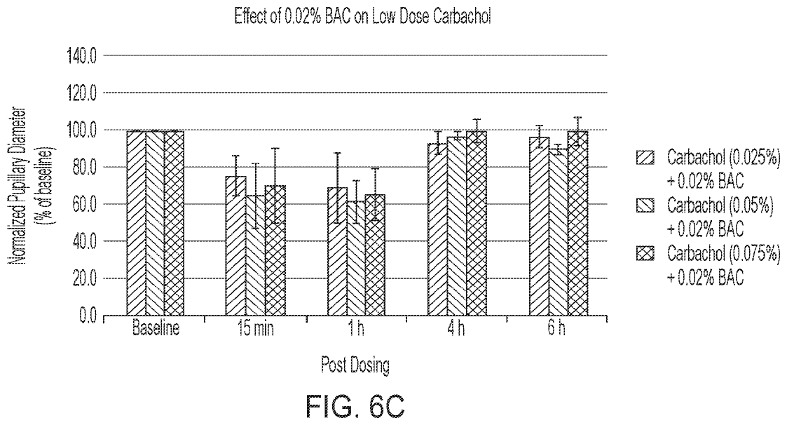

In still further experiments, the inventors investigated the effect of benzalkonium chloride on the miotic effect of carbachol. To that end, different concentrations of BAC were employed between 0.005% and 0.02% at carbachol concentrations between 0.025% and 0.075%, and the results are shown in FIGS. 6A-6C. More specifically, FIG. 6A shows the results for BAC at 0.005%, FIG. 6B results for BAC at 0.01%, and FIG. 6C shows the results for BAC at 0.005%. As can be readily seen from the graphs, BAC at a concentration of 0.005% had minimal effect with some effect seen at 1 hr at the higher carbachol concentration. The effect of BAC was somewhat more pronounced at a concentration of 0.01%, and the effect of BAC at a concentration of at least 0.02% was significant as is evident from the data in FIG. 6C. As BAK is believed to increase permeability of the respective agents across the corneal tissue, addition of BAK at concentrations up to 0.05% may advantageously increase miotic effect at otherwise identical drug concentration. Thus, BAK may enhance the miotic action of brimonidine, rivastigmine, galantamine, and carbachol, which may in turn allow for reduced drug concentrations and/or increased miotic effect without unduly extending the duration of the miotic effect.

Unless noted otherwise, all percentages indicated are weight percent (w/w). In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention. As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

1-60. (canceled)

61. A method of acute and transient treatment of night vision disturbance at a focal distance of at least 1 m in an individual, comprising:
topically administering an ophthalmic formulation to an eye of individual, wherein the ophthalmic formulation comprises
brimonidine in an amount of equal or less than 0.1%, and/or
rivastigmine in an amount of equal or less than 1.0%, and/or
galantamine in an amount of equal or less than 2.0%; and
wherein the acute and transient treatment results in an at least 10% reduction in pupillary diameter for between 1 hour and no more than 6 hours.

62. The method of claim 61, wherein the ophthalmic formulation comprises brimonidine, but not rivastigmine or galantamine.

63. The method of claim 61, wherein the ophthalmic formulation comprises rivastigmine, but not brimonidine or galantamine.

64. The method of claim 61, wherein the ophthalmic formulation comprises galantamine, but not brimonidine or rivastigmine.

65. The method of claim 61, wherein the ophthalmic formulation comprises brimonidine and rivastigmine, but not galantamine.

66. The method of claim 61, wherein the ophthalmic formulation comprises brimonidine and galantamine, but not rivastigmine.

67. The method of claim 61, wherein the ophthalmic formulation comprises brimonidine, rivastigmine, and galantamine.

68. The method of claim 61, wherein the brimonidine, if present in the ophthalmic formulation, is present in an amount of between 0.01% to 0.1%, wherein the brimonidine, if present in the ophthalmic formulation, is present in an amount of equal or less than 0.05%, and/or wherein the rivastigmine, if present in the ophthalmic formulation, is present in an amount of between 0.1% to 1.0%.

69. The method of claim 61, wherein the rivastigmine, if present in the ophthalmic formulation, is present in an amount of between 0.5% to 1.0%, wherein the galantamine, if present in the ophthalmic formulation, is present in an amount of between 0.2% to 2.0%, and/or wherein the galantamine, if present in the ophthalmic formulation, is present in an amount of between 0.5% to 1.5%.

70. The method of claim 61, wherein the ophthalmic formulation further comprises carbachol.

71. The method of claim 70, wherein the carbachol is present in the ophthalmic formulation in an amount of equal or less than 1.5%.

72. The method of claim 61, wherein the at least 10% reduction in pupillary diameter lasts for between 1 hour and no more than 4 hours.

73. The method of claim 61, wherein the acute and transient treatment results in an at least 15% reduction in pupillary diameter.

74. The method of claim 61, wherein the treatment reduces one or more than one of glare, starburst, halo, and double vision.

75. The method of claim 61, wherein the ophthalmic formulation further comprises benzalkonium chloride at an amount that increases delivery of brimonidine, rivastigmine, galantamine, and/or carbachol to the pupillary muscle.

76. The method of claim 61, wherein the ophthalmic formulation is sterile, and wherein the ophthalmic formulation is packaged in a single-use container or multi-dose container.

77. A topical ophthalmic formulation for acute and transient treatment of night vision disturbance, comprising:
brimonidine in an amount of equal or less than 0.1%, and/or
rivastigmine in an amount of equal or less than 1.0%, and/or
galantamine in an amount of equal or less than 2.0%; and
wherein, upon topical administration to an eye, the acute and transient treatment results in an at least 10% reduction in pupillary diameter and lasts for between 1 hour and no more than 6 hours.

78. The ophthalmic formulation of claim 77, wherein the brimonidine, if present in the ophthalmic formulation, is present in an amount of between 0.01% to 0.1%, wherein the rivastigmine, if present in the ophthalmic formulation, is present in an amount of between 0.1% to 1.0%, and/or wherein the galantamine, if present in the ophthalmic formulation, is present in an amount of between 0.2% to 2.0%.

79. The ophthalmic formulation of claim 77, wherein the ophthalmic formulation further comprises carbachol, and wherein the carbachol is present in the ophthalmic formulation in an amount of equal or less than 1.5%.

80. The ophthalmic formulation of claim 77, wherein the treatment reduces one or more than one of glare, starburst, halo, and double vision.
